(19) United States
(12) Patent Application Publication
(10) Pub. No.: US 2016/0121088 A1
(43) Pub. Date: May 5, 2016

(54) Devices and Methods for Dilating a Paranasal Sinus Opening and for Treating Sinusitis

(71) Applicant: SinuSys Corporation, Palo Alto, CA (US)

(72) Inventors: William Jason Fox, San Mateo, CA (US); William L. Gould, Santa Fe, NM (US); Todd A. Pelman, Moss Beach, CA (US)

(21) Appl. No.: 14/927,278

(22) Filed: Oct. 29, 2015

Related U.S. Application Data
(60) Provisional application No. 62/075,127, filed on Nov. 4, 2014.

(51) Int. Cl. A61M 29/00

(52) U.S. Cl. CPC A61M 29/00; A61M 2210/0681

(57) ABSTRACT

Medical devices which are adapted to be inserted into a patient for a limited period of time using minimally invasive insertion procedures for dilating a stenotic opening, such as a stenotic sinus opening, are provided. The devices and methods can be used for treating sinusitis and other nasal and/or sinus disorders.

### CROSS-REFERENCE TO RELATED APPLICATION

This application claims priority pursuant to 35 U.S.C. §119(e) to the filing date of U.S. Provisional Application No. 62/075127, filed Nov. 4, 2014, the disclosure of which is incorporated herein by reference.

### INTRODUCTION

The bones in the skull and face contain a series of air-filled cavities known as paranasal sinuses that are connected by passageways. The paranasal sinuses include frontal sinuses, sphenoid sinuses and maxillary sinuses. The paranasal sinuses are lined with mucus-producing epithelial tissue and are in communication with the nasal cavity. Normally, mucus produced by the epithelial tissue slowly drains out of each sinus through an opening known as an ostium. If the epithelial tissues lining a sinus cavity and/or sinus ostium become inflamed for any reason, the ostia and drainage of mucus therethrough can become blocked. This blockage can be periodic (resulting in episodes of pain) or chronic. This interference with drainage of mucus can result in mucosal congestion within the paranasal sinuses. Chronic mucosal congestion of the sinuses can cause damage to the epithelium that lines the sinus with subsequent decreased oxygen tension and microbial growth (e.g., a sinus infection).

The term “sinusitis” refers generally to any inflammation or infection of the paranasal sinuses caused by bacteria, viruses, fungi (molds), allergies or combinations thereof. It has been estimated that chronic sinusitis (e.g., lasting more than 3 months) results in 18 million to 22 million physician office visits per year in the United States. Patients who suffer from sinusitis typically experience at least some of the following symptoms: headaches or facial pain, nasal congestion or post-nasal drainage, difficulty breathing through one or both nostrils, bad breath and/or pain in the upper teeth. Thus, one of the ways to treat sinusitis is by restoring the lost mucus flow.

### SUMMARY

Medical devices which are adapted to be inserted into a patient for a limited period of time using minimally invasive insertion procedures for dilating a stenotic opening, such as a stenotic maxillary sinus opening or a stenotic sphenoid sinus opening, are provided. The devices and methods can be used for treating sinusitis and other nasal and/or sinus disorders.

In some embodiments, a device for dilating a stenotic opening of a paranasal sinus of a patient is provided. The device includes a self-expanding driver having an axis and configured to expand from a non-expanded configuration to an expanded configuration, wherein the non-expanded configuration is sized to be positioned within the stenotic sinus opening and wherein the device is sized to be introduced through a nostril opening and a nasal cavity of the patient in order to be positioned in the stenotic sinus opening. The device also includes an arm, extending from a proximal end of the device, for removably mounting the device on an insertion tool. The arm has an axis that approximately aligns with the nasal cavity and the nostril opening when the device is positioned in the stenotic sinus opening.

In certain embodiments, the stenotic paranasal sinus opening is a maxillary sinus opening, and the arm axis and the driver axis form an angle in the range of 90° to 130°. In certain embodiments, the angle is in the range of 100° to 120°, and in other embodiments, the angle is 110°. In certain embodiments, the arm has a sufficient length to prevent the device from passing completely through the stenotic opening and into the maxillary sinus.

In certain embodiments, the stenotic paranasal sinus opening is a sphenoid sinus opening, and the arm axis and the driver axis form an angle in the range of 0° to 30°. In certain embodiments, the angle is in the range of 5° to 25° and in other embodiments, the angle is 15°.

In certain embodiments, the arm has at least one retention element configured to prevent the arm from rotating when mounted on an insertion tool. In certain embodiments, the retention element is a ridge, a fin or a bump.

In certain embodiments, the device has a tether for ensuring that the device remains in place in the stenotic opening during treatment and for removing the device from the stenotic opening.

In certain embodiments, the driver is configured to expand the expandable portion from the non-expanded configuration to the expanded configuration over a period of 0.5 hours or more. In some embodiments, the driver is configured to expand the expandable portion from the non-expanded configuration to the expanded configuration over a period of 0.5 to 2 hours. In some embodiments, the driver is configured to expand the expandable portion by osmosis.

In certain embodiments, a kit is provided that includes the dilation device and a tool for inserting the device into a stenotic paranasal sinus opening of a patient. In certain embodiments, the tool has a cannula sized and configured to be removably coupled to the device by inserting at least a portion of the arm into the cannula.

Embodiments of the device may include a self-expanding driver having an axis and configured to expand from a non-expanded configuration to an expanded configuration, wherein the non-expanded configuration is sized to be positioned within the stenotic opening; and a proximal anchor comprising an arm extending radially from a proximal end of the device, the arm having a linear axis and being sized and configured to (i) be removably coupled to an insertion tool by inserting at least a portion of the arm into a cannula of said insertion tool, and (ii) prevent the device from passing completely through the stenotic opening and into the paranasal sinus, the driver axis and the arm axis forming an angle. In certain embodiments, the stenotic opening is a maxillary sinus opening, and the angle is in the range of 90° to 130°. In other embodiments, the stenotic opening is a sphenoid sinus opening and the angle is in the range of 0° to 30°.

### BRIEF DESCRIPTION OF THE FIGURES

FIG. 1 is a partial cutaway view of a human head showing the positions of the frontal sinuses (FS) and the maxillary sinuses (MS);

FIG. 2 is a sectional view of a portion of a human head showing the positions of the right frontal sinus (FS) and the right sphenoid sinus (SS);

FIG. 3 is a side view of an osmotically driven device for dilating a maxillary sinus opening, in a non-expanded configuration, according to embodiments of the present disclosure;

FIG. 4 is an end view of the device shown in FIG. 3;

FIG. 5 is a sectional view of the device shown in FIGS. 3 and 4, taken along line 5-5;

FIG. 6 is a side view of an osmotically driven device for dilating a sphenoid sinus opening, in a non-expanded configuration, according to embodiments of the present disclosure;

FIG. 7 is an end view of the device shown in FIG. 6;

FIG. 8 is a sectional view of the device shown in FIGS. 6 and 7, taken along line 8-8;

FIG. 9 is a perspective view of an insertion device having a maxillary sinus ostium dilation device mounted thereon, according to embodiments of the present disclosure;

FIG. 10 is a perspective view of an insertion device having a sphenoid sinus ostium dilation device mounted thereon, according to embodiments of the present disclosure;

FIG. 11 is an enlarged view of the distal end of the insertion device and dilator shown in FIG. 9;

FIG. 12 is an enlarged view of the distal end of the insertion device and dilator shown in FIG. 10;

FIG. 13 is a side view of portions of the insertion device and dilator device shown in FIG. 9;

FIG. 14 is a sectional view of the devices shown in FIG. 13;

FIG. 15 is a side view of portions of the insertion device and dilator device shown in FIG. 9 with trigger 203 in a distal position;

FIG. 16 is a sectional view of the devices shown in FIG. 15;

FIG. 17 is a side view of an insertion device and a dilation device used to insert the dilation device into the opening of a maxillary sinus which is shown in section, according to embodiments of the present disclosure;

FIG. 18 is a side view of the dilation device shown in FIG. 17 after being inserted into a maxillary sinus opening;

FIG. 19 is a side view of an insertion device and a dilation device used to insert the dilation device into the opening of a sphenoid sinus which is shown in section, according to embodiments of the present disclosure;

FIG. 20 is a side view of the distal end of an insertion device with a maxillary sinus ostium dilator mounted thereon, according to embodiments of the present disclosure;

FIG. 21 is a side view of the distal end of an insertion device with a maxillary sinus ostium dilator mounted thereon, according to U.S. Application Publication No. 2013/0231693; and

FIG. 22 is a side view of the insertion device and dilator shown in FIG. 21, with the positions of the device and dilator shown at the time of dilator deployment.

Before embodiments of the present disclosure are described in greater detail, it is to be understood that these embodiments are not limited to the particular aspects described, and as such may, of course, vary. It is also to be understood that the terminology used herein is for the purpose of describing particular embodiments only, and is not intended to be limiting, since the scope of the embodiments is embodied by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within embodiments of the present disclosure. The upper and lower limits of these smaller ranges may independently be included in the smaller ranges and are also encompassed within the embodiments, subject to any specifically excluded limit in the stated range. Where the stated range includes one or both of the limits, ranges excluding either or both of those included limits are also included in the embodiments.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present embodiments, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, it will be readily apparent to one of ordinary skill in the art in light of the teachings herein that certain changes and modifications may be made thereto without departing from the spirit and scope of the appended claims. Any recited method can be carried out in the order of events recited or in any other order which is logically possible.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. To the extent such publications may set out definitions of a term that conflict with the explicit or implicit definition of the present disclosure, the definition of the present disclosure controls. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

### DETAILED DESCRIPTION

Medical devices which are adapted to be inserted into a patient for a limited period of time using minimally invasive insertion procedures for dilating a stenotic opening, such as a stenotic sinus opening, are provided. The devices and methods can be used for treating sinusitis and other nasal and/or sinus disorders.

### Devices and Methods for Dilating a Stenotic Opening of a Paranasal Sinus in a Subject

Aspects of the present disclosure include devices and methods for dilating a stenotic opening of a paranasal sinus in a subject. The device (also referred to herein as a dilator, a sinus dilator, a dilation device, or a dilator device) includes an expandable portion configured to expand from a non-expanded configuration to an expanded configuration, where the non-expanded configuration is sized to be positioned within the stenotic opening, and a driver configured to expand the expandable portion from the non-expanded configuration to the expanded configuration, where the expanded configuration dilates the stenotic opening.

The term “stenotic opening” refers to an abnormal narrowing of a biological passageway, such as a paranasal sinus opening. In certain embodiments, the device includes an osmotic driver configured to expand an expandable portion from a non-expanded configuration to an expanded configuration, and the expandable portion disposed peripherally around the driver and configured to expand from the non-expanded configuration to the expanded configuration, where the non-expanded configuration is sized to be positioned within the stenotic opening.

In certain embodiments, the driver is self-expanding when in contact with tissue of the subject. By “self-expanding” is meant that the driver may expand from the non-expanded configuration to the expanded configuration without external intervention from a user or a health care practitioner. For example, the self-expanding driver may be self-contained, such that the driver is configured to expand without connection to an external pressure source. As such, self-expanding drivers as described herein function without the need for an external pressure source or a pressure monitoring device (e.g., as with a balloon catheter). In some cases, the self-expanding driver expands from the non-expanded configuration to the expanded configuration upon absorbing fluid from the surrounding environment when the device is in use. For instance, the self-expanding driver may expand from the non-expanded configuration to the expanded configuration upon absorbing water from the surrounding tissues of the stenotic opening when the device is in use. Self-expanding drivers may be configured to expand the expandable portion of the device by various ways, such as, but not limited to, an osmotic agent, a swellable agent (e.g., a swellable polymer), combinations thereof, and the like. In some instances, the driver is configured to expand the expandable portion by at least one of osmosis, a shape memory metal, a spring, a swellable polymer, a thermal expansion of a gas, a thermal expansion of a liquid, a gas-generating chemical reaction and a phase change expansion of a material.

In certain embodiments, the driver includes an osmotic agent. As used herein, the terms “osmotic agent,” “osmotically active agent” and “osmoagent” are used interchangeably and refer to an agent that facilitates the imbibition of water from a region of high water potential (e.g., low solute concentration) through a semipermeable membrane to a region of low water potential (e.g., high solute concentration) until a state of dynamic equilibrium is reached. In some instances, the osmotically active agent may be configured to absorb water flowing through a semipermeable membrane from the surrounding tissues after insertion of the device into the stenotic opening of the subject and expand. In certain embodiments, the osmotic agent is configured to have a zero order rate of expansion. By “zero order” is meant that the rate of volume expansion of the osmotic agent is approximately constant over time and is independent of the surrounding solute concentration. In certain embodiments, the osmotically active agent is a salt (e.g., a water-soluble salt), such as, but not limited to sodium chloride.

In certain embodiments, the driver includes an osmopolymer, such as a hydrogel-forming osmopolymer (e.g., an osmopolymer that forms a hydrogel when exposed to water). In some instances, the osmopolymer is a water-soluble polymer, such as, but not limited to a polyethylene oxide polymer (e.g., Polyox 303; Dow Chemical Company).

In certain embodiments, the driver is configured to begin expanding upon insertion of the device into the stenotic opening of the subject. The terms “insert” or “insertion” are used herein interchangeably to describe the positioning of a device in a stenotic opening of a subject for a period of time. In some instances, the driver is configured to begin expanding within seconds or minutes after insertion of the device into the stenotic opening. In some cases, the driver is configured to begin expanding in 60 minutes or less, such as 45 minutes or less, or 30 minutes or less, including 10 minutes or less, or 5 minutes or less, such as 1 minute or less, after insertion of the device into the stenotic opening. In some instances, the driver is configured to continue to expand for a certain period of time after the device has been inserted into the stenotic opening of the subject. For example, the driver may be configured to continue to expand for 30 minutes or more, such as 45 minutes or more, including 60 minutes or more, or 90 minutes or more, 120 minutes or more, or 180 minutes or more, or 240 minutes or more, or 300 minutes or more after the device has been inserted into the stenotic opening of the subject.

In certain embodiments, the driver takes a certain amount of time to expand the expandable portion from the non-expanded configuration to the expanded configuration. For instance, in some cases the driver is configured to expand the expandable portion from the non-expanded configuration to the expanded configuration over a period of 0.5 hours or more, such as 1 hour or more, or 2 hours or more, or 4 hours or more, or 6 hours or more, or 8 hours or more, or 10 hours or more, or 12 hours or more, or 24 hours or more, or 48 hours or more, or 72 hours or more, etc. In some instances, the driver is configured to expand the expandable portion from the non-expanded configuration to the expanded configuration over a period of 24 hours or less, such as 12 hours or less, or 10 hours or less, or 8 hours or less, or 6 hours or less, or 4 hours or less, or 2 hours or less, 1.5 hours or less, or 1 hour or less, or 0.5 hour or less. As such, in certain instances, the driver is configured to expand the expandable portion from the non-expanded configuration to the expanded configuration over a period ranging from 0.5 hour to 24 hours, such as 0.5 hour to 12 hours, including 0.5 hour to 10 hours, or 1 hour to 8 hours, or 1 hour to 6 hours, or 1 hour to 4 hours, or 1 hour to 2 hours.

In certain embodiments, the driver is configured to expand the expandable portion to a diameter of 10 mm or less, such as 9 mm or less, or 8 mm or less, or 7 mm or less, or 6 mm or less, or 5 mm or less, or 4 mm or less, or 3 mm or less, or 2 mm or less, or 1 mm or less. In some cases, the driver is configured to expand the expandable portion to a diameter of 7 mm or less.

In certain embodiments, the driver is configured to exert an expansion pressure on the stenotic opening of 5 psi (35 kPa) or more, or 10 psi (70 kPa) or more, or 15 psi (105 kPa) or more, or 20 psi (140 kPa) or more, or 25 psi (170 kPa) or more, or 30 psi (210 kPa) or more, or 35 psi (240 kPa) or more, or 40 psi (275 kPa) or more, or 45 psi (310 kPa) or more, or 50 psi (345 kPa) or more. In some instances, the driver is configured to exert an expansion pressure on the stenotic opening of 50 psi (345 kPa) or less, or 45 psi (310 kPa) or less, or 40 psi (275 kPa) or less, or 35 psi or less, or 30 psi (210 kPa) or less, or 25 psi (170 kPa) or less, or 20 psi (140 kPa) or less, or 15 psi (105 kPa) or less, or 10 psi (70 kPa) or less, or 5 psi (35 kPa) or less. As such, in some cases, the driver is configured to exert an expansion pressure on the stenotic opening ranging from 5 psi (35 kPa) to 50 psi (345 kPa), such as 5 psi (35 kPa) to 45 psi (310 kPa), or 5 psi (35 kPa) to 40 psi (275 kPa), or 5 psi (35 kPa) to 35 psi (240 kPa), or 10 psi (70 kPa) to 30 psi (210 kPa), or 15 psi (105 kPa) to 25 psi (170 kPa). In certain embodiments, the driver is configured to exert an expansion pressure on the stenotic opening of 20 psi (140 kPa).

As used herein, the term “distal” refers to the end of a device (e.g., a sinus dilator device or insertion device), or a component thereof, that is positioned towards the end of the device that is inserted through or closest to a paranasal sinus opening of the subject. For example, the distal end of a sinus dilator device is the end of the device that is inserted through the paranasal sinus opening of the subject and remains within the sinus cavity during use. A device (e.g., a sinus dilator device or insertion device), or a component thereof, may also include a proximal end. As used herein, the term “proximal” refers to the end of the device, or a component thereof, that is positioned towards the end of the device that remains on the nasal cavity side of the stenotic opening or remain external to the subject during use. For example, the proximal end of a sinus dilator device is the end of the device that remains on the nasal cavity side of the stenotic opening when the sinus dilator device is positioned in the stenotic opening during use.

Embodiments of the presently disclosed devices include an expandable portion. The expandable portion is configured to expand from a non-expanded configuration to an expanded configuration. In certain embodiments, the expandable portion is configured to expand in size from a non-expanded configuration to an expanded configuration. The expandable portion may be configured to expand in size without significantly increasing in volume, such as by stretching in one or more dimensions from the non-expanded configuration. The expandable portion may be positioned peripherally around the driver. For instance, the expandable portion may be disposed on an exterior surface of the driver. In these embodiments, expansion of the underlying driver expands the expandable portion from its non-expanded configuration to its expanded configuration.

Aspects of the present disclosure include devices that have an expandable portion, where the expandable portion includes a membrane. The membrane may be an elastic membrane, such that the membrane is configured to expand from the non-expanded configuration to the expanded configuration, as described herein. In certain instances, the membrane is a semipermeable membrane. By “semipermeable” is meant a membrane that is permeable to solvent but not significantly permeable to solute across a concentration gradient, such as a membrane that allows solvent (e.g., water) molecules to pass through the membrane by osmosis from a region of low solute concentration to a region of high solute concentration until a state of dynamic equilibrium is reached. For instance, a semipermeable membrane may be configured to allow water to pass through the membrane by osmosis from a region of low solute concentration (e.g., high water potential) to a region of high solute concentration (e.g., low water potential) until a state of dynamic equilibrium is reached.

In certain embodiments, the expandable portion includes a membrane, where the membrane is an impermeable membrane. By “impermeable” is meant a membrane that is not significantly permeable to solvent or solute. Impermeable membranes do not allow significant amounts of solvent (e.g., water) or solute molecules to pass through the membrane by osmosis even in the presence of a solute concentration gradient across the membrane.

In certain embodiments, the driver is disposed on an exterior surface of a tube or conduit. The driver may be disposed on the exterior surface of the conduit at a position between the distal end and the proximal end of the conduit. For example, the driver may be positioned between a tapered tip at the distal end of the conduit and a mounting arm at the proximal end of the conduit. As described herein, the expandable portion may be positioned peripherally around the driver. Thus, in these embodiments, the driver is disposed between the exterior surface of the conduit and the overlying expandable portion. Expansion of the driver expands the overlying expandable portion from its non-expanded configuration to its expanded configuration.

Aspects of the driver further include embodiments where the driver completely surrounds the conduit. The driver may be disposed on the exterior surface of the conduit around the entire periphery of the conduit. In certain embodiments, the driver surrounds the conduit around the central portion of the conduit, where the proximal end of the conduit may have a mounting arm extending therefrom and the distal end of the conduit may have a tapered tip, as described in more detail herein. In some instances, the driver includes one or more subunits, where each subunit is disposed on the exterior surface of the conduit. The one or more driver subunits may be positioned such that they are in contact with the adjacent one or more driver subunits.

In certain embodiments, the walls of the conduit are substantially rigid. The walls of the conduit may be substantially rigid, such that the conduit maintains substantially the same shape and size during use of the device. For instance, the conduit may maintain substantially the same interior diameter during use of the device. In some instances, the walls of the conduit are substantially rigid, such that pressure exerted on the exterior surface of the conduit by the driver does not significantly decrease the interior diameter of the conduit. For example, the walls of the conduit may be substantially rigid, such that the conduit is not crushed by the driver during use of the device. In some instances, the driver is configured to expand radially outward from the conduit. As discussed above, the walls of the conduit may be substantially rigid, thus expansion of the driver may be directed radially outward away from the substantially rigid walls of the conduit. Expansion of the driver radially outward from the conduit may facilitate dilation of the stenotic opening.

In certain embodiments, the walls of the conduit are substantially non-collapsible. The walls of the conduit may be substantially non-collapsible, such that the conduit is configured to maintain an opening in the conduit during use of the device. For example, the walls of the conduit may be substantially non-collapsible, such that the conduit is not crushed by the driver during use of the device. In some cases, a non-collapsible conduit maintains substantially the same shape and size during use of the device. For instance, the conduit may maintain substantially the same interior diameter during use of the device. In some instances, the walls of the conduit are substantially non-collapsible, such that pressure exerted on the exterior surface of the conduit by the driver does not significantly decrease the interior diameter of the conduit. As discussed above, the driver may be configured to expand radially outward from the conduit and, as such, the walls of the conduit may be substantially non-collapsible, such that expansion of the driver is directed radially outward away from the substantially non-collapsible walls of the conduit. Expansion of the driver radially outward from the conduit may facilitate dilation of the stenotic opening. A substantially non-collapsible conduit may be rigid, as described above, or may be flexible and adapted to bend from its original shape. In some instances, a flexible conduit facilitates insertion of the sinus dilator in a sinus ostium.

In certain instances, the conduit includes a membrane. The conduit membrane may be a semipermeable membrane. In certain instances, the conduit membrane is a non-collapsible semipermeable membrane. In some cases, the conduit membrane is a rigid semipermeable membrane. The membrane may be configured to be permeable to solvent but not significantly permeable to solute across a concentration gradient, such that the membrane allows solvent (e.g., water) molecules to pass through the membrane by osmosis from a region of low solute concentration to a region of high solute concentration until a state of dynamic equilibrium is reached. For instance, the membrane may be configured to allow water to pass through the membrane by osmosis from an interior lumen of the conduit to the surrounding driver until a state of dynamic equilibrium is reached.

In some embodiments, the device includes a conduit that includes a semipermeable membrane, a surrounding driver, and an overlying expandable portion that includes a semipermeable membrane. In these embodiments, the device may be configured to allow solvent (e.g., water) to pass through both the semipermeable expandable portion membrane by osmosis and through the semipermeable conduit membrane by osmosis. For example, the device may be configured to allow solvent to pass through the semipermeable expandable membrane from the surrounding tissues to the underlying driver, and also allow solvent to pass through the semipermeable conduit membrane from an interior lumen of the conduit to the surrounding driver.

In other embodiments, the device includes a conduit that includes a semipermeable membrane, a surrounding driver, and an overlying expandable portion that includes an impermeable membrane. In these embodiments, the device may be configured to allow solvent (e.g., water) to pass through the semipermeable conduit membrane by osmosis but not allow significant amounts of solvent (e.g., water) to pass through the impermeable expandable portion membrane. For example, the device may be configured to allow solvent to pass through the semipermeable conduit membrane from an interior lumen of the conduit to the surrounding driver, but not allow significant amount of solvent to pass through the impermeable expandable portion membrane to the driver.

In yet other embodiments, the conduit includes an impermeable material. In some cases, the impermeable material is an impermeable membrane. For instance, the device may include a conduit that includes an impermeable membrane, a surrounding driver, and an overlying expandable portion that includes a semipermeable membrane. In these embodiments, the device may be configured to allow solvent (e.g., water) to pass through the semipermeable expandable membrane by osmosis but not allow significant amounts of solvent (e.g., water) to pass through the impermeable conduit membrane. For example, the device may be configured to allow solvent to pass through the semipermeable expandable portion membrane from the surrounding tissues to the underlying driver, but not allow significant amount of solvent to pass through the impermeable conduit membrane from the interior lumen of the conduit to the surrounding driver.

Aspects of the device may include a distal anchor configured to maintain the device within the stenotic opening during use of the device. The distal anchor may be connected to the device proximate to the distal end of the device. For example, the distal anchor may be connected to the device proximate to the distal end of the conduit. In some cases, the distal anchor is configured to prevent the device from premature explantation from the stenotic opening. The distal anchor may facilitate maintaining the device within the stenotic opening for a desired period of time until the device is removed from the stenotic opening by the user or a health care professional. In certain embodiments, the distal anchor is a mechanical anchor, such as, but not limited to, a hook, a barb, a clamp, a tether and the like. In certain cases, the distal anchor is configured to maintain the device within the stenotic opening by having a diameter that is greater than the diameter of the stenotic opening.

In some instances, the device has a frictional surface on an exterior surface of the device. The frictional surface may be configured to increase the friction between the exterior surface of the device and the surrounding tissues when the device is in use. Increasing the friction between the exterior surface of the device and the surrounding tissues may facilitate retention of the device in the stenotic opening of the subject during use. For example, the frictional surface may have a rough topography that includes an exterior surface shaped as, for example, washboard, rings, waffle pattern, snow tire pattern, pebble finish, shark skin texture, combinations thereof, and the like.

In certain cases, the device includes an adhesive disposed on an exterior surface of the device. In some cases, the membrane includes an adhesive. The membrane may be configured such that the adhesive elutes to the external surface of the device during use. The adhesive may facilitate retention of the device in the stenotic opening of the patient during use. Examples of suitable adhesives include, but are not limited to, carbomer, low molecular weight hydroxypropyl methylcellulose, polyvinyl pyrrolidone, combinations thereof, and the like.

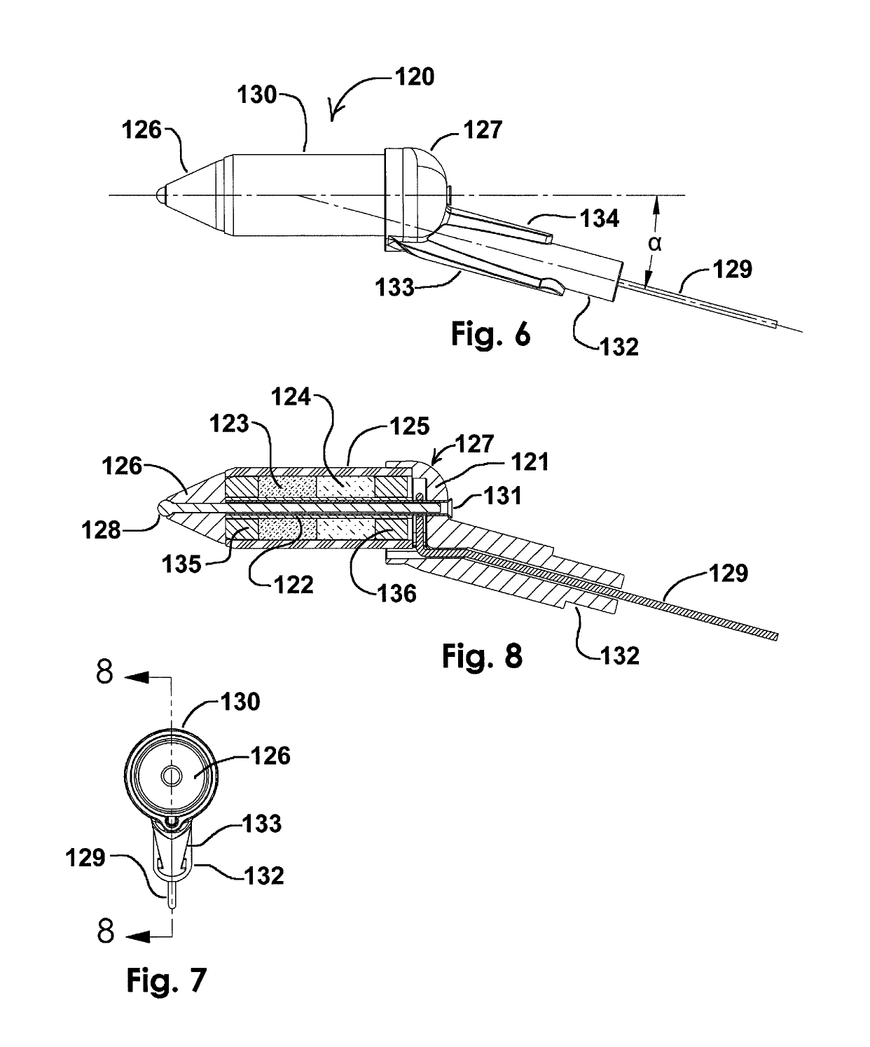

In some cases, the distal anchor is configured to allow the device to be inserted into the stenotic opening. The distal anchor may have an outside diameter that is substantially the same as the outside diameter of the device when the device is in a non-expanded configuration. In some instances, the distal anchor has an outside diameter that is greater than the diameter of the conduit. In certain embodiments, the distal anchor has a tapered shape, such that the distal end of the distal anchor has a diameter that is less than the diameter of the proximal end of the distal anchor (see e.g., FIGS. 5 and 6 of US Patent Publication US 2013/0231693 published Sep. 5, 2013). In certain embodiments, the distal anchor is configured such that the distal anchor has a diameter that is smaller during insertion of the device into the stenotic opening as compared to the diameter of the distal anchor after the anchor portion of the device has been inserted into the paranasal sinus.

Aspects of the device may include a mounting arm (also referred to herein as an arm or a mounting member) at the proximal end of the device. As described in more detail below, the mounting arm may be configured to facilitate attachment of the device to an insertion tool for inserting the device into a sinus opening of a subject.

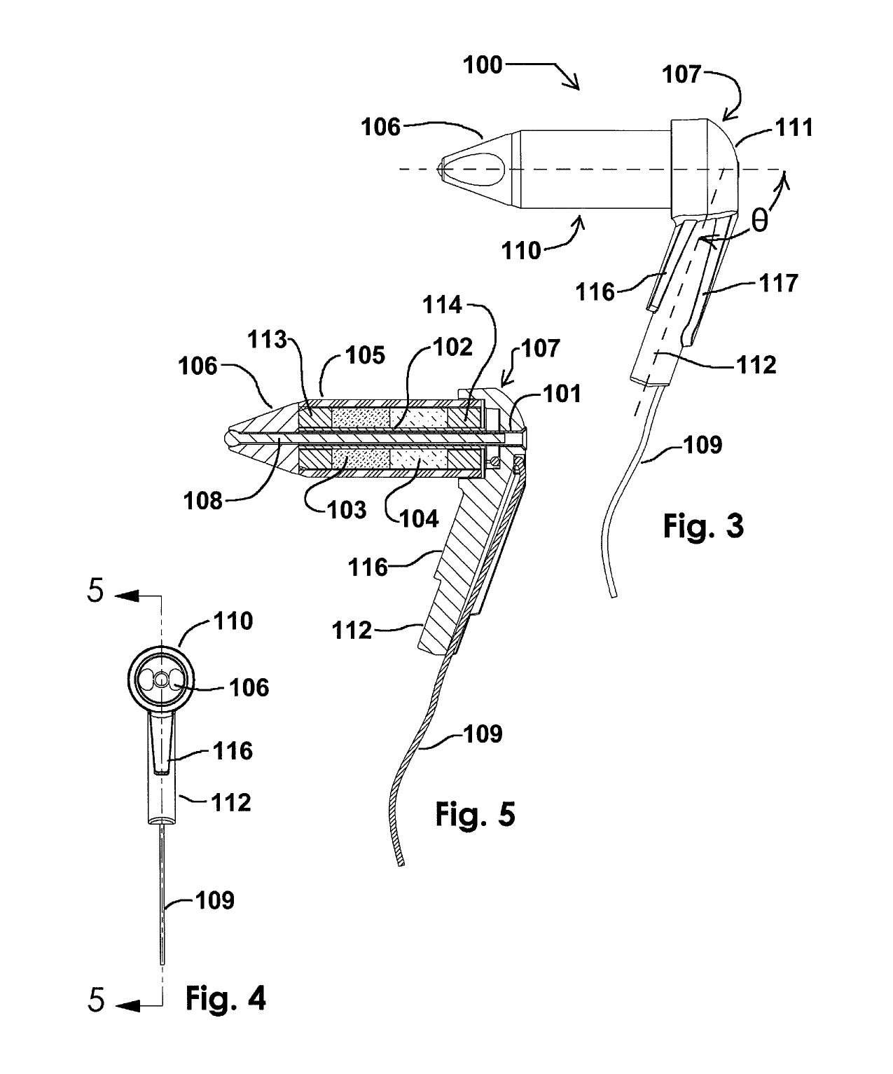

In some instances, the mounting arm may extend radially outward from an axis (e.g., a longitudinal axis) of the device. The arm may have a length sufficient to prevent the device from entering further into the sinus cavity of the subject, thus keeping the device properly positioned within the stenotic sinus opening of the subject during use. As such, the mounting arm may also function as a proximal anchor, where the proximal anchor is configured to maintain the device within the stenotic opening during use of the device (see e.g., FIGS. 3-5 and 18). The proximal anchor may be connected to the device proximate to the proximal end of the device. For example, the proximal anchor may be connected to the device proximate to the proximal end of the conduit. In some cases, the proximal anchor is configured to prevent the device from being inserted too far or completely into the paranasal sinus of the subject. The proximal anchor may facilitate maintaining the device within the stenotic opening for a desired period of time until the device is removed from the stenotic opening by the user or a health care professional. In some cases, the proximal anchor/mounting arm may extend radially outward from the longitudinal axis of the device with a length such that the maximum overall width of the device (e.g., diameter of the dilator plus height of the mounting arm extending outward) is greater than the diameter of the sinus ostium. For instance, the proximal anchor may extend radially outward from the longitudinal axis of the device with a length such that the maximum overall width of the device (e.g., diameter of the dilator plus height of the mounting arm extending outward) is greater than the diameter of the sinus ostium when the device is in an expanded configuration.

In some embodiments, the device includes an attachment portion configured to facilitate removal of the device from the stenotic opening. The attachment portion may be configured to allow a removal device to be attached to the device. For example, the attachment portion of the device may include a structure, such as, but not limited to, a loop, a tether or a hook. The removal device may include a corresponding structure that allows for attachment of the removal device to the attachment portion of the device. In some instances, the device includes a loop and the removal device includes a hook. In other embodiments, the device includes a hook and the removal device includes a loop. In either embodiment, insertion of the hook into the loop connects the device to the removal device and may facilitate removal of the device from the stenotic opening. I some instances, the attachment portion is a tether having a length sufficient for a free end of the tether to extend out of a patient's nostril when the device is placed in the stenotic opening. Such a tether also has the advantage of enabling the surgeon to determine if the dilator remains in place in the stenotic opening during treatment, for instance by giving a slight tug on the tether to see if the dilator has pushed out of the ostium and is merely sitting loose within the nasal cavity.

In some cases, the attachment portion may protrude from the device to facilitate connection of the removal device to the attachment portion of the device. The attachment portion may be disposed at or near the proximal end of the device to facilitate removal of the device from the stenotic opening. For example, the attachment portion may be disposed on or adjacent to the proximal arm at the proximal end of the device. In certain cases, the attachment portion may be connected to the conduit proximate to the proximal end of the device.

Additional aspects of the devices and methods for dilating a stenotic opening of a paranasal sinus in a subject are described in more detail in U.S. Application Publication Nos. 2012/0053567 and 2012/0053404, both published Mar. 1, 2012, and U.S. Application Publication No. 2013/0231693, published Sep. 5, 2013, the disclosures of each of which are incorporated herein by reference.

### Devices and Methods for Inserting a Sinus Dilator

Aspects of the present disclosure include an insertion device adapted to insert a sinus dilator into a stenotic opening of a paranasal sinus in a subject patient using minimally invasive insertion procedures. The insertion device and methods can be used to treat sinusitis and other nasal and/or sinus disorders. The insertion device may also be referred to herein as an insertion tool (e.g., a tool for inserting a sinus dilator into a stenotic opening of a patient).

The insertion device includes a handheld member coupled to a hollow elongated member. By “hollow” is meant that the hollow elongated member includes a central passageway that extends through the length of the hollow elongated member. For example, the hollow elongated member may be a tube or a cannula. In certain embodiments, the proximal end of the hollow elongated member may be coupled to a handheld member and the distal end of the hollow elongated member is dimensioned to pass through a nasal cavity of a subject. A sinus dilator, as described above, may be coupled to the distal end of an insertion device, which may then be inserted into the nasal cavity of a subject. The distal end of the hollow elongated member may include a retention interface that removably couples to a sinus dilator. The sinus dilator may be coupled to the retention interface (e.g., slid in, snapped on, clamped on, etc.) and then the distal end of the insertion device may be inserted within the nasal cavity to position the sinus dilator within the stenotic opening. In certain embodiments, the retention interface and sinus dilator are configured to be removably coupled, thus the sinus dilator may be decoupled from the insertion device and left within the stenotic opening. The sinus dilator is then positioned within a stenotic sinus opening, which may be partially or completely occluded.

In certain embodiments, the insertion device also includes an interior elongated member positioned within the hollow elongated member and extending at least a portion of the length of the hollow elongated member. For example, the interior elongated member may be a rod. The interior elongated member has a proximal end coupled to the handheld member and dimensioned to fit within the hollow elongated member.

The retention interface may include various coupling mechanisms to retain the sinus dilator coupled to the insertion device. In some instances, the retention interface is a cannula having an open distal end sized and shaped to allow the arm of a sinus dilator to fit therewithin. The retention interface may provide sufficient retention to maintain the sinus dilator coupled while permitting some light axial and off-axis loads or bending moments. In some instances, the sinus dilator is sufficiently rigidly affixed to the retention interface to enable a user (e.g., physician) to push the sinus dilator through a stenotic opening even when the opening is completely shut.

The portions of the insertion device that are advanced through a nostril opening in order to access a stenotic sinus opening may be in the form of a straight cannula having an open distal end for mounting a sinus dilator thereon, e.g., by sliding an arm of the sinus dilator into the open distal end. The distal end of the cannula may be slotted, and the arm of the dilator may have correspondingly positioned fins, ridges or bumps, etc., to provide a retention element whereby the dilator is unable to rotate around the axis of the arm once mounted on the end of the cannula. In some instances, at least a portion of the arm (e.g., the proximal end of the arm) has cross-sectional shape that matches the cross-sectional shape of the cannula of the insertion tool. For example, the arm may have a circular cross-section for slidably mounting on the cannula of the insertion tool as described above. In some cases, the arm and the cannula have concentric circular cross-sections, for example such that a portion of the proximal end of the arm can be inserted into the open distal end of the cannula of the insertion tool.

As summarized above, the insertion device also includes a handheld member. As the handheld member is held by the user, it is configured to have a shape and size that is amenable to gripping by the user's hand. The insertion device may include, for example, a trigger that is located in a position for the user to actuate the trigger in order to decouple a sinus dilator coupled to the distal end of the insertion device. For instance, the insertion device may be shaped and sized to be gripped by a physician's hand with the trigger accessible to the user's hand while gripping the handheld member, e.g., actuated by the physician's thumb, actuated by a user's index finger (for instance, with a gun-like trigger), etc. The trigger may, for example, be configured to couple to the interior elongated member or hollow elongated member. It should be appreciated that an electrical circuit can be created to actuate the mechanical translation of the interior elongated member or hollow elongated member.

Upon activation of the trigger, the retention interface is decoupled from the sinus dilator. For example, the interior elongated member may be relatively displaced with respect to the hollow elongated member or cannula. In some embodiments, the relative displacing of the interior elongated member with respect to the hollow elongated member includes proximally displacing the retention interface within the hollow elongated member while the hollow elongated member/cannula remains in a substantially fixed position relative to the handheld member. In some cases, actuation of trigger in the embodiments described above decouples the sinus dilator from the insertion device as the mounting arm of the sinus dilator is displaced distally with respect to the hollow elongated member.

In other embodiments, the relative displacing of the interior elongated member with respect to the hollow elongated member includes distally pushing the arm out of the hollow elongated member while the hollow elongated member remains in a substantially fixed position relative to the handheld member. In some instances, the distal tip of the interior elongated member may push against the mounting arm of the sinus dilator as the interior elongated member is displaced distally within the hollow elongated member. In some cases, actuation of the trigger in the embodiments described above decouples the sinus dilator from the insertion device as the distal tip of the interior elongated member is displaced distally with respect to the hollow elongated member. For example, the interior elongated member may be configured to push the arm of the sinus dilator (e.g., the proximal end of the arm) out of the hollow elongated member (i.e., cannula) and thus decouple the sinus dilator from the insertion device when the interior elongated member is displaced distally within the hollow elongated member (i.e., cannula).

The overall weight of the insertion device may take into account usability as a handheld device by the user, e.g., to permit a physician to easily hold and handle the device during an insertion procedure. The shape of the handheld member may vary, but in some instances may be in the shape of a wand with a button or switch trigger, a gun-like handle and trigger, or other graspable and usable shape.

As summarized above, the insertion device is dimensioned such that at least the distal end of the device can pass through the nasal cavity of a subject. The distal end may include, for example, at least a portion of the hollow elongated member, interior elongated member and retention interface. As such, at least the distal end of the device has a cross-sectional diameter that is 10 mm or less, such as 8 mm or less, and including 5 mm or less. The elongated members may have the same outer cross-sectional dimensions (e.g., diameter) along its entire length. Alternatively, the cross-sectional diameter may vary along the length of the elongated members.

Furthermore, the lengths of the hollow elongated member and interior elongated member may vary. For example, the lengths of the elongated members may vary depending on the specific sinus being targeted. In some instances, the lengths of the elongated members range from 1 cm to 20 cm, such as 2 cm to 15 cm, including 5 cm to 10 cm. It should be appreciated that in some instances the hollow elongated member and interior elongated member may have different lengths from one another.

As stated above, the hollow elongated member and interior elongated member of the insertion device has a proximal end and a distal end. The term “proximal end”, as used herein, refers to the end of the elongated members (or the insertion device or other component on the insertion device) that are nearer the user (such as a physician operating the device in an insertion procedure), and the term “distal end”, as used herein, refers to the end of the elongated members (or the insertion device or other component on the insertion device) that are nearer the target stenotic opening of the subject during use.

The hollow elongated members may be, for example, a structure of sufficient rigidity to allow the distal end of the sinus dilator to be pushed through tissue when sufficient force is applied to the proximal end of the insertion device. As such, in some embodiments, the elongated member is not pliant or flexible, at least not to any significant extent. Example materials may include, but are not limited to, metals, metal alloys (e.g., stainless steel), polymers such as hard plastics, etc.

In some embodiments, to facilitate access to an opening of the maxillary sinus, the axis of the sinus dilator driver and the axis of the sinus dilator mounting arm form an angle ranging from 90° to 130°, such as 100° to 120°, including 105° to 115°, or 110°. In some embodiments, to facilitate access to an opening of the sphenoid sinus, the axis of the sinus dilator driver and the axis of the sinus dilator mounting arm form an angle ranging from 0° to 30°, such as 10° to 20°, or 15°.

The interior elongated member or rod may be, in some instances, a structure of sufficient rigidity to allow the sinus dilator to be pushed through the stenotic opening when sufficient force is applied to the proximal end of the device, even when the stenotic opening is completely occluded. In some instances, the interior elongated member may be a metal, metal alloy, polymer (hard or pliant and flexible), etc.

In some embodiments, the retention interface includes retaining elements that provide an additional securing force to the sinus dilator so that it may not slide back off the retention interface unless a sufficient amount of force is applied to overcome the additional securing force, or until the additional securing force is removed. For example, the retention interface, for instance, include a compressible lip, bump, or other protrusion on the mounting arm of the dilator that is compressed when inserted within the open end of the cannula of the insertion device, e.g., lips, bumps or protrusion that fit within mating recesses on the cannula that “snap” the dilator onto the distal end of the cannula. In some instances, the distal tip of the cannula is split (e.g., in a polymer flexure design), with each arm of the split tip stressed or flexed outward away from one another when the mounting arm of the sinus dilator is inserted between the arms of the split tip.

It should also be appreciated that the above described retaining elements are exemplary and that other types of retaining elements may be implemented. It should also be appreciated that the retaining element described above, and equivalents thereof, serve as means for providing an additional securing force to the sinus dilator when inserted on the retention interface.

In some embodiments, the insertion device may be configured to include a camera positioned near the distal end of the hollow elongated member in order to assist in visualizing the stenotic site, nasal cavity, or sinus cavity. In some instances, the camera may be positioned on the exterior surface of the hollow elongated member and, for example, electrically coupled to a monitor via an electrical wire extending along or within the hollow elongated member. In other instances, the camera may be positioned within the hollow elongated member. For example, a camera may be positioned at the tip of the interior elongated member and electrically coupled to a monitor via an electrical wire extending within the interior elongated member.

The insertion device, or components thereof, may be configured for one time use (i.e., disposable) or may be re-usable, e.g., where the components are configured to be used two or more times before disposal, e.g., where the device components are sterilizable.

### Additional Aspects of the Sinus Dilator and Insertion Device

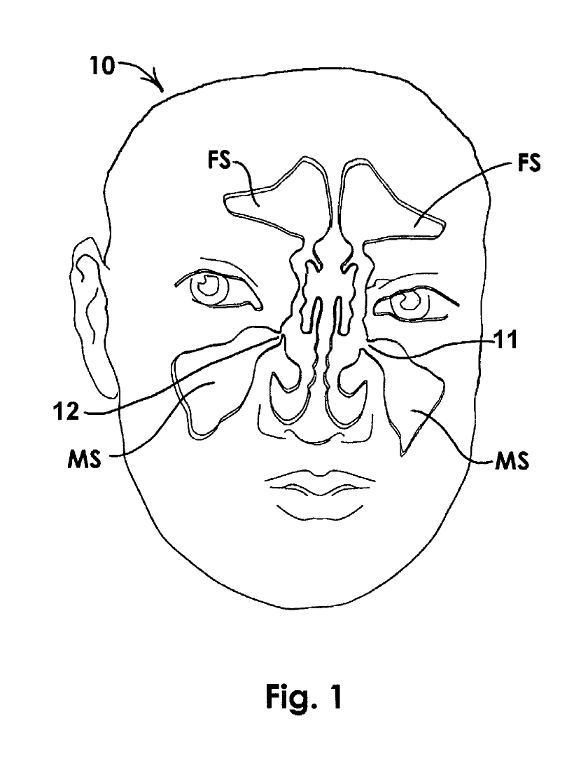

Referring now to FIG. 1, there is shown a human patient 10 having two frontal sinuses (FS) and two maxillary sinuses (MS). Each of these four sinuses has an opening which can be accessed by way of the patient's nostrils. The openings include maxillary sinus openings 11 and 12, of which opening 11 is shown in a normal open condition and opening 12 shown in an occluded or stenotic condition.

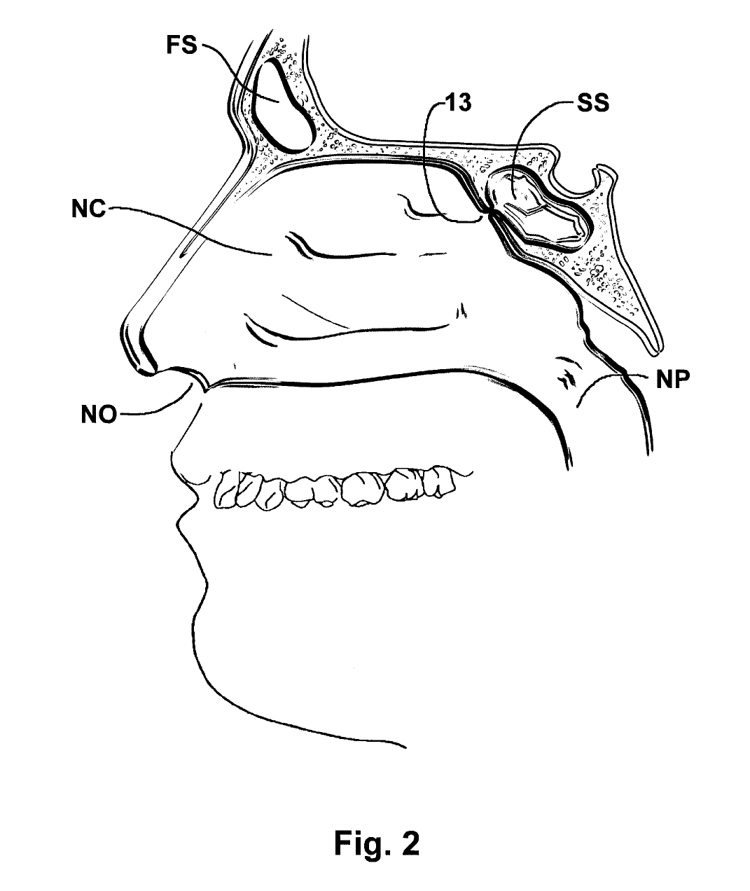

Referring now to FIG. 2, there is shown a sectional view of a patient's nose and sinuses including the nasal cavity (NC), the nasopharynx (NP), the nostril opening (NO), the frontal sinus (FS), the sphenoid sinus (SS) and the sphenoid sinus opening 13 which is shown in a stenotic condition prior to any dilation procedure.

Additional aspects of the sinus dilator and the insertion device will now be described in more detail below.

In certain embodiments, the device includes a mounting arm (also referred to herein as an arm or a mounting member) at the proximal end of the device. In certain embodiments, the mounting arm extends radially outward from an axis (e.g., a longitudinal axis) of the device. The arm may have a length sufficient to prevent the device from entering further into the sinus cavity of the subject, thus keeping the dilator properly positioned within the stenotic sinus opening of the subject during use. A device configured in such a manner may facilitate a sinus dilator that does not include a distal anchor (e.g., the distal anchor being the anchor positioned inside the sinus cavity).

One embodiment of an osmotic maxillary sinus ostium dilator 100 which illustrates several aspects of the present disclosure is shown in a side view in FIG. 3, in end view in FIG. 4, and in a sectional view in FIG. 5. The dilator 100 includes an osmotic driver 110, a tapered distal tip 106, a proximal end piece 107 which is comprised of a proximal end cap 111 and a mounting member 112 (also referred to herein as a “mounting arm” or an “arm”). Distal tip 106 can be made of a soft and flexible material (e.g., a rubber such as polyether block amide) to assist in pushing the dilator 100 through a stenotic maxillary sinus ostium with minimal damage to the ostial tissues.

As shown in FIG. 3, the axes of osmotic driver 110 and mounting member 112 are shown as dashed lines. For the maxillary sinus dilator 100, the angle θ formed between these two lines ranges, in certain embodiments from 90° to 130°, in other embodiments from 100° to 120°, in other embodiments from 105° to 115°, and in other embodiments is about 110°.

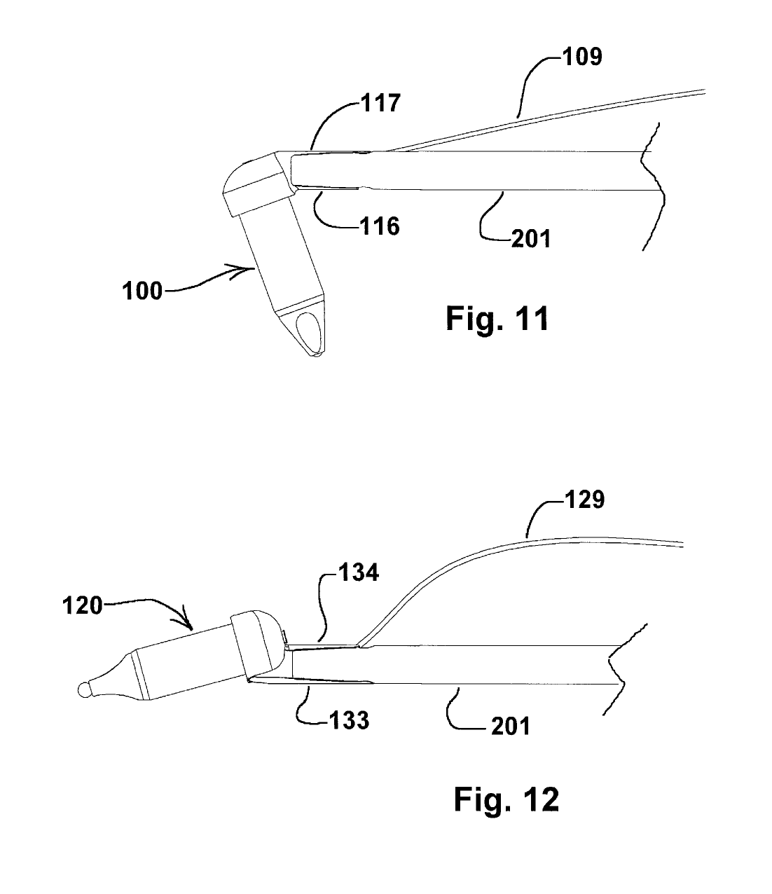

As shown in FIGS. 3 and 4, mounting member 112 has raised wedge-shaped fins 116 and 117 (also referred to herein as retention elements) which are sized and shaped to slide into corresponding slots in the distal end of the dilator insertion tool, and prevent rotation of the dilator around the axis of the mounting member 112 when the dilator 100 is mounted on the insertion tool (see, e.g., FIG. 11).

Attached to the end piece 107 is a tether 109 which can be in the form of a string or polymeric (e.g., Kevlar or nylon) line. Tether 109 is long enough to extend out through the patient's nostril once the dilator 100 is placed in the patient's maxillary sinus ostium. The tether 109 is used to pull the dilator out of the sinus ostium and the nasal cavity after the dilation procedure is completed. End piece 107 is attached to the osmotic driver 110, for example using adhesive, a mechanical coupling, welding, or forming end piece 107 and mounting member 112 as a single unit. Optionally after attaching the end piece 107 to the driver 110, the proximal end of tube 101 is flared to further lock the end piece 107 to the driver 110.

Dilator 100 is shown in a non-expanded configuration in FIGS. 3 to 5. Dilator 100 includes tube 101 (e.g., a non-collapsible metal or plastic tube) having the osmotic driver 110 disposed thereon. As shown in FIG. 5, the driver 110 is comprised of an inner membrane 102 disposed on the tube 101, two annularly-shaped osmotic tablets 103, 104 threaded over the membrane 102 and tube 101, two annularly-shaped spacers 113 and 114 and an elastic semipermeable membrane 105 applied over the osmotic tablets and the spacers. The spacers 113 and 114 may be formed of the same type of polymer used in membranes 102 and 105 so that there is good bonding therebetween. Inside of tube 101 is a pin 108 having an enlarged distal end. The pin 108 can be made of stainless steel or a hard plastic and is attached to tube 101, for example using a suitable adhesive, interference fit (e.g., press fit or friction fit), detent, knurl, etc. The pin 108 functions to lock the distal tip 106 to the osmotic driver 110.

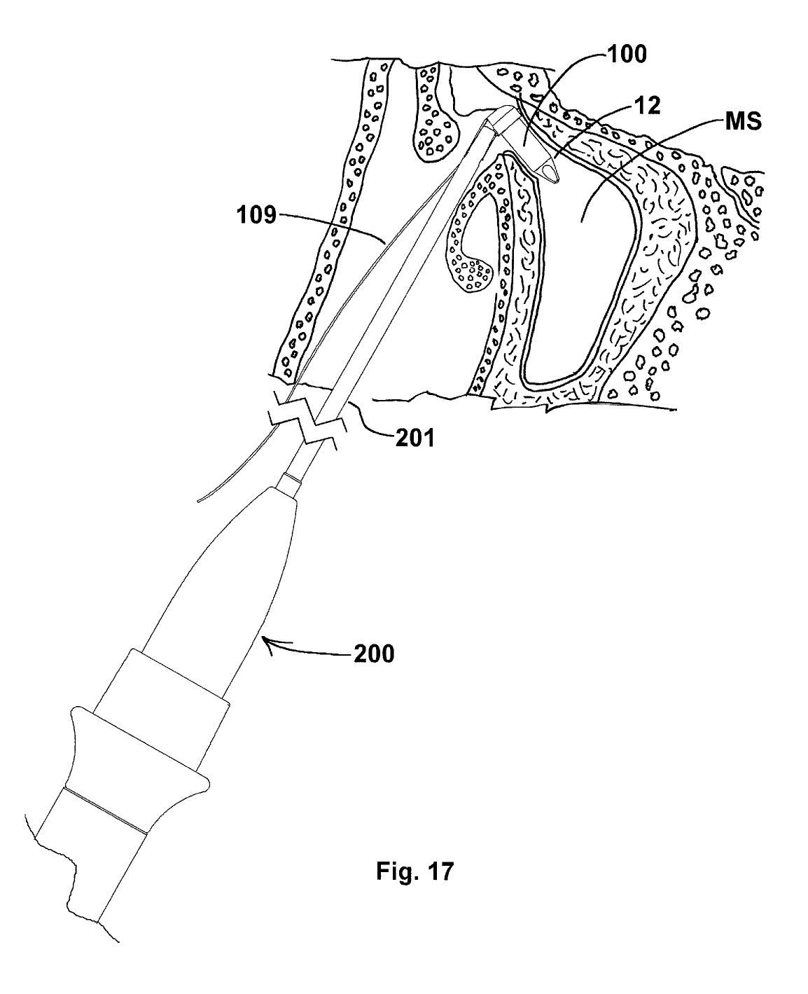

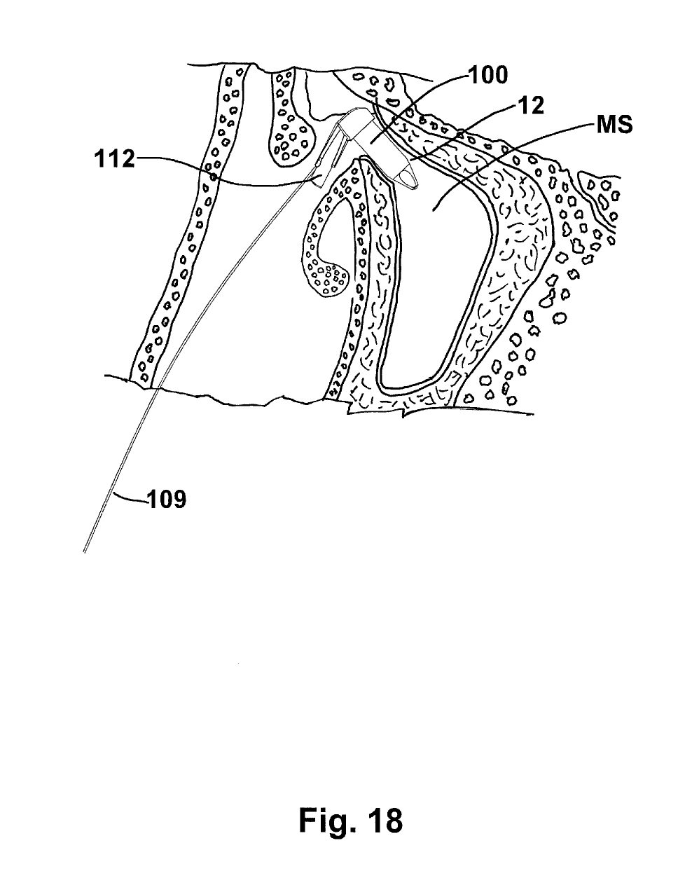

As shown in FIGS. 17 and 18, in use, a maxillary sinus dilator 100 is placed in the maxillary sinus opening of a living subject, typically a human subject. Water from the subject's body and tissues permeates through the elastic semipermeable membrane 105 due to the presence of an osmotic pressure difference caused by the osmotically active agent(s) contained in tablets 103 and 104. As water permeates into the tablets 103, 104, they begin to swell. Since tube 101 is made of an incompressible material (e.g., stainless steel) and since end piece 107 and tip 106 are also made from relatively incompressible materials (e.g., plastic, metal or ceramic) and are locked together via the pin 108 and the flared proximal end of tube 101, the swelling of tablets 103, 104 causes the device 100 to expand primarily in a radially outward direction, and not in an axial direction. In other words, the diameter of osmotic driver 110 increases. The swelling tablets 103, 104 cause the elastic membrane 105 to expand to accommodate the increasing volume of the tablets 103, 104. As disclosed in greater detail in U.S. Application Publication No. 2012/0053567, published Mar. 1, 2012, the disclosure of which is incorporated herein by reference, the expanding osmotic driver 110 exerts pressure on the surrounding tissue and bone of the sinus opening, causing the opening to permanently dilate. By controlling membrane 105 thickness and composition, as well as osmotic tablet 103, 104 composition, the period for complete expansion of the driver 110 is at least 0.5 hours and in certain embodiments is about 1 hour. In certain embodiments, expansion over a period of at least 0.5 hours is desirable since it avoids patient discomfort and tissue damage experienced with abrupt short-term dilation times as are encountered in balloon sinuplasty procedures. In those applications where the dilation procedure using the dilators disclosed herein occurs in a physician's office setting while the subject is awake and waiting, dilation typically occurs over a period of less than 2 hours, though longer dilation times may optionally be used.

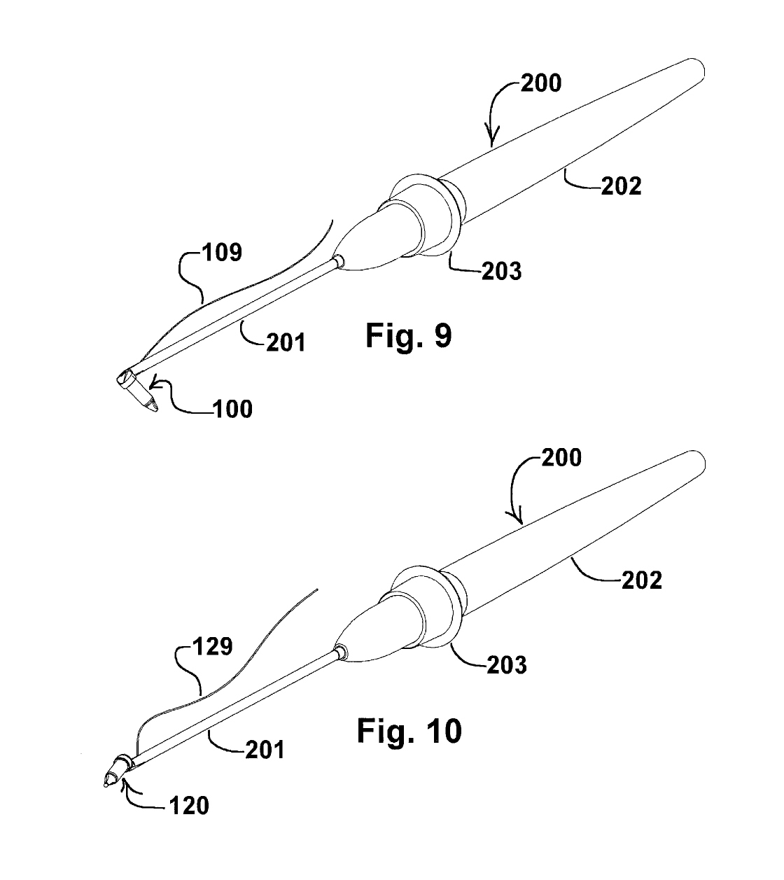

In certain embodiments, the insertion of a dilator 100 into a stenotic maxillary sinus opening is as follows. Referring first to FIGS. 9, 11 and 13, there is shown one embodiment of a sinus ostium dilator insertion device 200 having a maxillary sinus ostium dilator mounted on the distal end thereof. Device 200 has a handle 202, a cannula 201 mounted on the distal end of handle 202, the handle 202 having a slidable trigger 203. The cannula 201 is substantially straight along its entire length and the mounting member 112 of the dilator 100 (see, e.g., FIG. 3) slides into the distal open end of the cannula 201. The distal tip of cannula 201 has slots therein which are sized to slidably engage the fins 116 and 117 of mounting member 112 such that the fins 116 and 117 extend out of the slots of cannula 201 and prevent rotation of the dilator 100 around the axis of mounting member 112 during insertion of dilator 100 into a stenotic maxillary sinus opening (see also FIGS. 3-5).

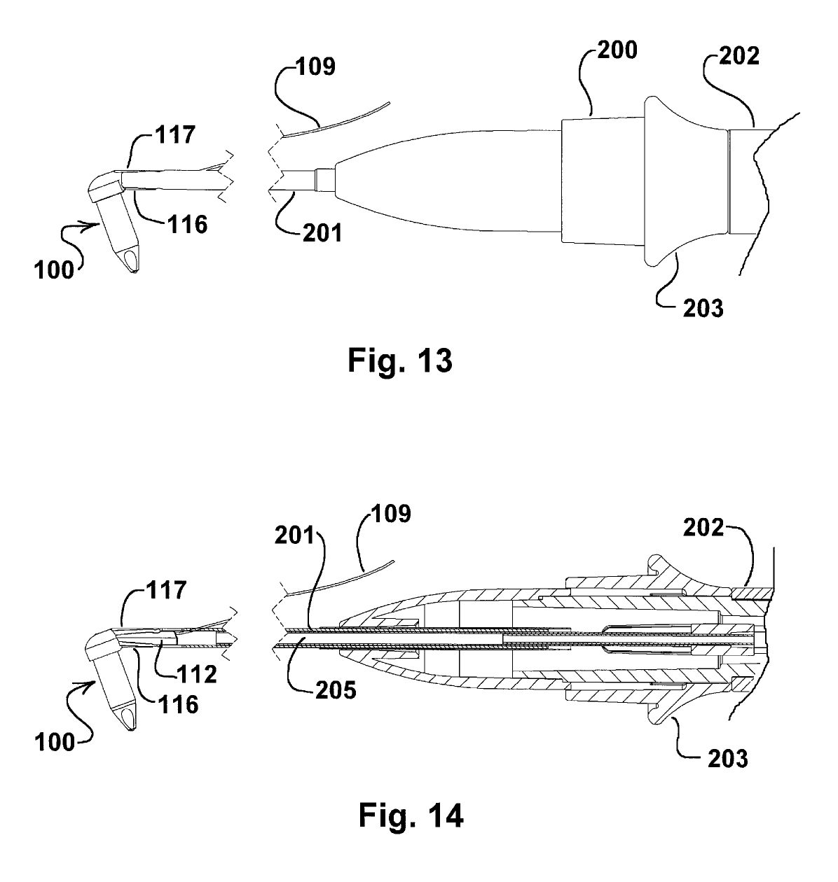

Referring now to FIGS. 13 and 14, with dilator 100 mounted onto the distal end of device 200, the sliding trigger 203 is in its proximal position. The trigger 203 is connected to rod 205 via conventional mechanical connection means through a slot in handle 202. Rod 205 is slidably positioned within cannula 201. The rod 205 can be for example made from metal or plastic and has a diameter just slightly less than the inner diameter of cannula 201. The proximal end of rod 205 is mechanically connected to trigger 203 so that when trigger 203 is slid in a distal direction, rod 205 also moves within cannula 201 in a distal direction, and when trigger 203 is slid in a proximal direction, rod 205 also moves within cannula 201 in a proximal direction. In other embodiments, mechanical manipulation of rod 305 with trigger 203 may be achieved by, for example rotation, push-in (e.g., push-in in a radial direction), and the like. With the sliding trigger 203 oriented in the proximal position (e.g., the right position as shown in FIGS. 13 and 14), the dilator 100 is mounted on the distal end of insertion device 200 and is ready for deployment into a sinus opening. In this position, the distal end of rod 205 is recessed a sufficient distance from the distal end of cannula 201 to accommodate mounting member 112 to fit within the interior lumen of cannula 201.

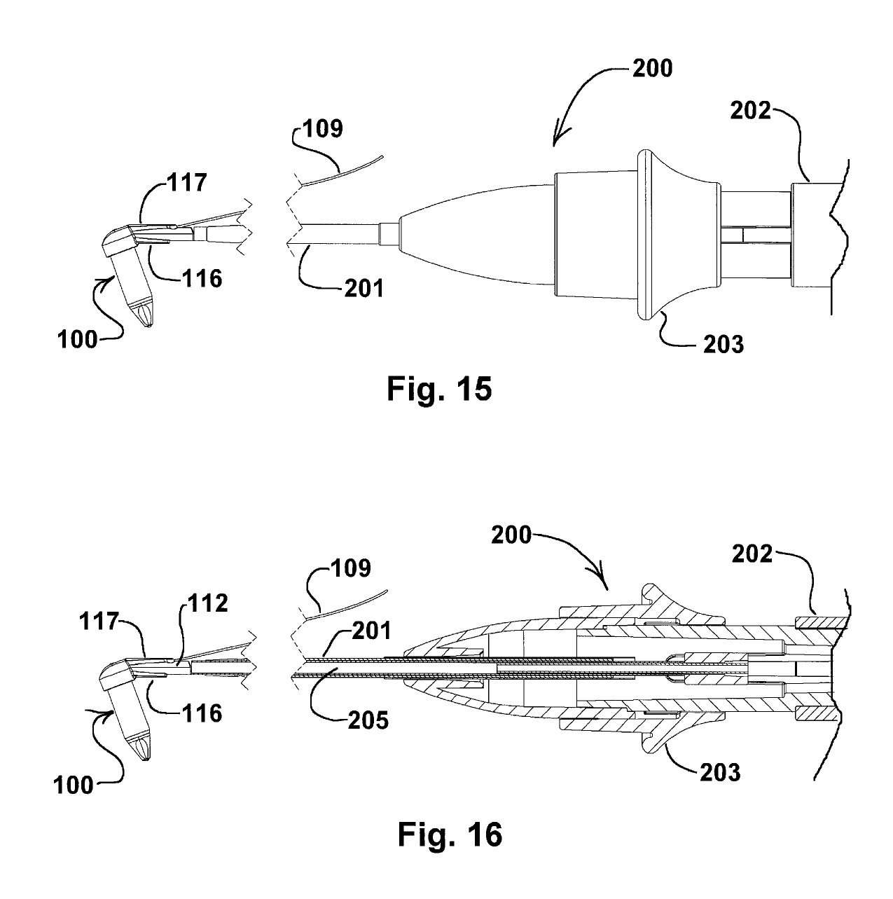

In use, and as shown in FIGS. 15, 16, 17 and 18, the mounted dilator 100, and cannula 201 are advanced through the subject's nostril and then through the nasal passageway, until the distal tip of dilator 100 abuts against the stenotic opening. Then the physician applies further force on handle 202 and pushes the dilator 100 into the sinus opening 12 until the cannula 201 abuts against the tissue surrounding the nasal passageway side of sinus opening 12. Because the fins 116 and 117 fit snugly within corresponding slots in cannula 201, the dilator 100 can be pushed into a narrowed, stenotic and/or completely closed opening by the physician applying a distally and/or radially oriented (i.e., radially in relation to the cannula 201 axis) pushing force via the handle 202. As shown in FIGS. 17 and 18, the mounting member 112 (e.g., also referred to herein as a mounting arm or an arm) has an axis that approximately aligns with the nasal cavity and the nostril opening when the device is positioned in the maxillary sinus opening 12.

Once in position within the sinus opening 12, the physician may slide the trigger 203 to the distal position (e.g., the left position as shown in FIGS. 15 and 16), and the rod 205 is advanced distally in the interior lumen of cannula 201 which causes the dilator 100 to be pushed off the distal end of cannula 201. Following, the insertion device 200 is withdrawn from the patient by moving the device 200 in a direction that is roughly parallel to the nasal cavity.

As shown in FIG. 18, the mounting member 112 acts as an anchor to prevent the dilator 100 from being inadvertently pushed too far into the maxillary sinus cavity. After insertion, the dilator 100 remains in the sinus ostium and expands over a period of time, e.g., 0.5 to 2 hours. Once fully expanded, or at a later time if more convenient for the physician or the patient, the dilator is removed from the enlarged ostium using either forceps or by pulling on tether 109.

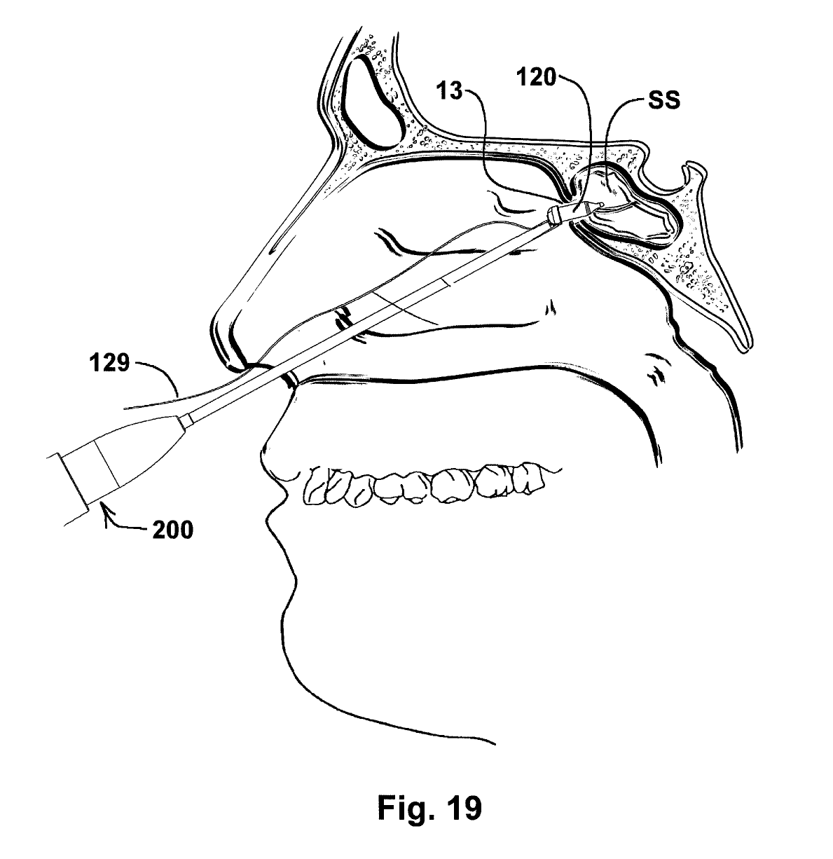

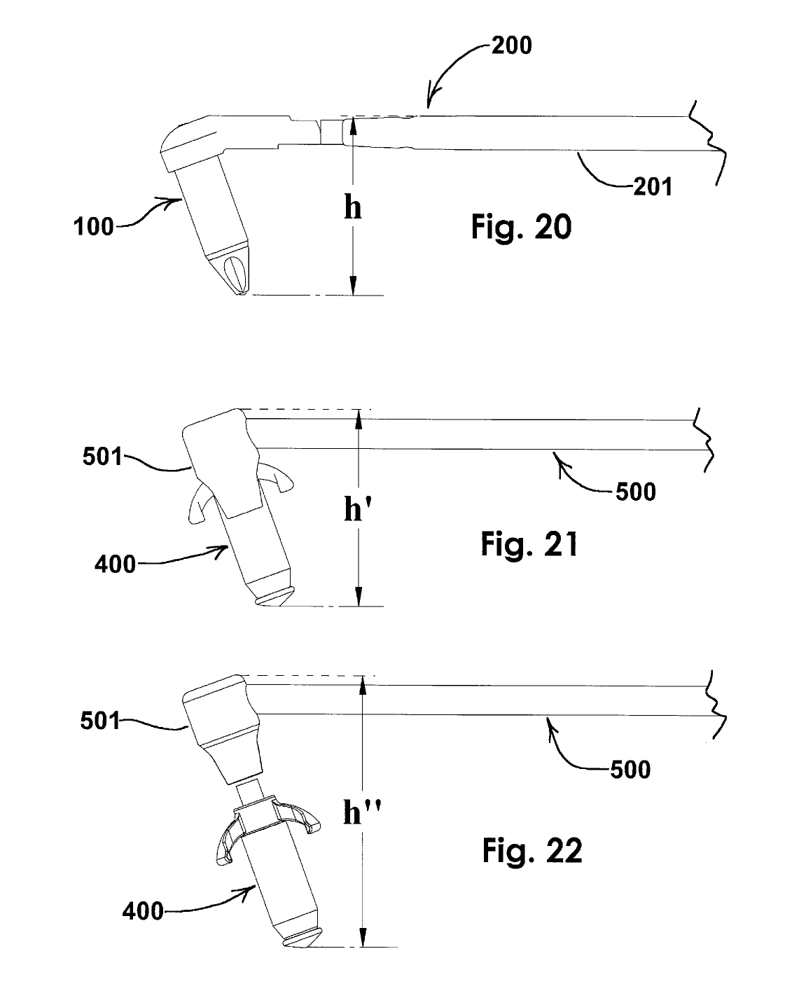

The insertion device 200 has a similarly sized low profile distal end as that shown in FIG. 19 of U.S. Application Publication No. 2013/0231693 published Sep. 5, 2013. The distal end portion of the insertion device described in U.S. 2013/0231693 is shown in side views in FIGS. 21 and 22. As a side-by-side comparison, FIG. 20 shows the distal end of the insertion device 200 according to embodiments described herein with a maxillary sinus dilator 100 mounted thereon, whereas FIG. 21 shows the distal end of insertion device 500 with a maxillary sinus dilator 400 mounted thereon. Dilators 100 and 400 each have a length of about 13 mm and they are each mounted on the distal end of their respective insertion devices so that the longitudinal axes of the dilators 100 and 400 are each at a 110° angle (θ) with respect to the axis of the hollow elongated member (e.g., cannula 201) of their respective insertion devices 200 and 500. As shown in FIG. 20, the height (h) of the insertion device 200 and mounted dilator 100 may be measured from the upper surface of the cannula 201 to the distal tip of the sinus dilator 100.

While the mounted dilator and insertion device shown in FIG. 21, has a similar height (h′), in order to deploy dilator 400 from insertion device 500, the insertion device 500 must be backed away from the dilator 400 a sufficient distance to allow the proximal end of dilator 400 to clear the mounting flange 501 as shown in FIG. 22. Thus, in order to release dilator 400 from insertion device 500, there must be a height h″ provided within the nasal cavity adjacent the maxillary sinus ostium. In some cases, the human anatomy in that portion of a human nasal cavity may present a restrictive space, making deployment of dilator 400 challenging. In embodiments of the present disclosure, as shown in FIG. 20, dilator 100 is deployed by pushing dilator 100 off the end of cannula 201 as described above, and backing cannula 201 out through the nasal cavity. Deployment of dilator 100 is easier and simpler compared to deployment of dilator 400. Thus, the smaller height h compared to height h″ facilitates deployment of dilator 100 in the maxillary sinus ostium after insertion thereof.

Another embodiment of an osmotic paranasal sinus ostium dilator is a sphenoid sinus ostium dilator 120 which illustrates several aspects of the present disclosure and is shown in a side view in FIG. 6, in end view in FIG. 7, and in a sectional view in FIG. 8. The dilator 120 includes an osmotic driver 130, a tapered distal tip 126, a proximal end piece 127 which is comprised of a proximal end cap 121 and a mounting member 132 (also referred to herein as a “mounting arm” or an “arm”). Distal tip 126 can be made of a soft and flexible material (e.g., a rubber such as polyether block amide) to assist in pushing the dilator 120 through a stenotic sphenoid sinus ostium with minimal damage to the ostial tissues.

As shown in FIG. 6, the axes of osmotic driver 130 and mounting member 132 are shown as dotted lines. For the sphenoid sinus dilator 120, the angle a formed between these two lines ranges, in certain embodiments from 0° to 30°, in other embodiments from 5° to 25°, in still other embodiments from 10° to 20°, and in another embodiment is about 15°.

As shown in FIGS. 6 and 7, mounting member 132 has raised wedge-shaped fins 133 and 134 (also referred to herein as retention elements) which are sized and shaped to slide into corresponding slots in the distal end of the dilator insertion tool, and prevent rotation of the dilator around the axis of the mounting member 132 when the dilator 120 is mounted on the insertion tool (see, e.g., FIG. 12).

Attached to the end piece 127 is a tether 129 which can be in the form of a string or polymeric (e.g., Kevlar or nylon) line. Tether 129 is long enough to extend out through the patient's nostril once the dilator 120 is placed in the patient's sphenoid sinus ostium. The tether 129 is used to pull the dilator out of the sinus ostium and the nasal cavity after the dilation procedure is completed. End piece 127 is attached to the osmotic driver 130, for example using adhesive, a mechanical coupling, welding, or forming end piece 127 and mounting member 132 as a single unit. Optionally after attaching the end piece 127 to the driver 130, the proximal end of tube 131 is flared to further lock the end piece 127 to the driver 130.

Dilator 120 is shown in a non-expanded configuration in FIGS. 6 to 8. Dilator 120 includes tube 131 (e.g., a non-collapsible metal or plastic tube) having the osmotic driver 130 disposed thereon. As shown in FIG. 8, the driver 130 is comprised of an inner membrane 122 disposed on the tube 131, two annularly-shaped osmotic tablets 123, 124 threaded over the membrane 122 and tube 131, two annularly-shaped spacers 135 and 136 and an elastic semipermeable membrane 125 applied over the osmotic tablets and the spacers. The spacers 135 and 136 may be formed of the same type of polymer used in membranes 122 and 125 so that there is good bonding therebetween. Inside of tube 131 is a pin 128 having an enlarged distal end. The pin 128 can be made of stainless steel or a hard plastic and is attached to tube 131, for example using a suitable adhesive, interference fit (e.g., press fit or friction fit), detent, knurl, etc. The pin 128 functions to lock the distal tip 126 to the osmotic driver 130.

As shown in FIG. 19, in use, a sphenoid sinus dilator 120 is placed in the sphenoid sinus opening of a living subject, typically a human subject. Water from the subject's body and tissues permeates through the elastic semipermeable membrane 125 due to the presence of an osmotic pressure difference caused by the osmotically active agent(s) contained in tablets 123 and 124. As water permeates into the tablets 123, 124, they begin to swell. Since tube 131 is made of an incompressible material (e.g., stainless steel) and since end piece 127 and tip 126 are also made from relatively incompressible materials (e.g., plastic, metal or ceramic) and are locked together via the pin 128 and the flared proximal end of tube 131, the swelling of tablets 123, 124 causes the dilator 120 to expand primarily in a radially outward direction, and not in an axial direction. In other words, the diameter of osmotic driver 130 increases. The swelling tablets 123, 124 cause the elastic membrane 125 to expand to accommodate the increasing volume of the tablets 123, 124. As disclosed in greater detail in U.S. Application Publication No. 2012/0053567 published Mar. 1, 2012, the disclosure of which is incorporated herein by reference, the expanding osmotic driver 130 exerts pressure on the surrounding tissue and bone of the sinus opening, causing the opening to permanently dilate. By controlling membrane 125 thickness and composition, as well as osmotic tablet 123, 124 composition, the period for complete expansion of the driver 130 is at least 0.5 hours and in certain embodiments is about 1 hour. In certain embodiments, expansion over a period of at least 0.5 hours is desirable since it avoids patient discomfort and tissue damage experienced with abrupt short-term dilation times as are encountered in balloon sinuplasty procedures. In those applications where the dilation procedure using the dilators disclosed herein occurs in a physician's office setting while the subject is awake and waiting, dilation typically occurs over a period of less than 2 hours, though longer dilation times may optionally be used.

In certain embodiments, the insertion of a dilator 120 into a stenotic sphenoid sinus opening is as follows. Referring first to FIGS. 10 and 12, there is shown one embodiment of a sinus ostium dilator insertion device 200 having a sphenoid sinus ostium dilator 120 mounted on the distal end thereof. The insertion device 200 used to insert dilator 120 into a stenotic sphenoid sinus ostium is the same as the device 200 described earlier herein used to insert dilator 100 into a maxillary sinus ostium. Thus one advantage of the present dilators 100 and 120 is that they can be used with the same insertion device 200. This is accomplished by designing the angle of the mounting member relative to the driver differently in the maxillary and sphenoid dilators, yet having the same size and shape mounting member, and one which is adapted to mount on the open end of a straight, non-curved cannula 201.

As shown in FIG. 12, the distal tip of cannula 201 has slots therein which are sized to slidably engage the fins 133 and 134 of mounting member 132 such that the fins 133 and 134 extend out of the slots of cannula 201 and prevent rotation of the dilator 120 around the axis of member 132 during insertion of dilator 120 into a stenotic sphenoid sinus opening (see also FIGS. 6-8).

Referring now to FIGS. 13 and 14, with dilator 100 mounted onto the distal end of device 200, the sliding trigger 203 is in its proximal position. The trigger 203 is operatively connected to rod 205 as described earlier herein through a slot in handle 202. Rod 205 is slidably positioned within cannula 201. The rod 205 can be for example made from metal or plastic and has a diameter just slightly less than the inner diameter of cannula 201. The proximal end of rod 205 is operatively connected to trigger 203. With the sliding trigger 203 oriented in the proximal position (e.g., the right position as shown in FIGS. 13 and 14), the dilator 100 is mounted on the distal end of insertion device 200 and is ready for deployment into a sinus opening. In this position, the distal end of rod 205 is recessed a sufficient distance from the distal end of cannula 201 to accommodate mounting member 112 to fit within the interior lumen of cannula 201. The description above regarding FIGS. 13 and 14 in relation to dilator 100 also applies to dilator 120 because, as discussed above, the same configuration of the insertion device 200 may be used for either dilator 100 or dilator 120.

In use, and as shown in FIG. 19, the mounted dilator 120, and cannula 201 are advanced through the subject's nostril and then through the nasal passageway, until the distal tip of dilator 120 abuts against the stenotic sphenoid sinus opening 13. Then the physician applies further force on handle 202 and pushes the dilator 120 into the sinus opening 13. Because the fins 133 and 134 fit snugly within corresponding slots in cannula 201, the dilator 120 can be pushed into a narrowed, stenotic and/or completely closed opening by the physician applying a distally oriented pushing force via the handle 202. As shown in FIG. 19, the mounting member (e.g., also referred to herein as a mounting arm or an arm) of dilator 120 has an axis that approximately aligns with the nasal cavity and the nostril opening when the device is positioned in the sphenoid sinus opening 13.

Once in position within the sinus opening 13, the physician may slide the trigger 203 to the distal position (e.g., the left position as shown in FIGS. 15 and 16), and the rod 205 is advanced distally in the interior lumen of cannula 201 which causes the dilator 120 to be pushed off the distal end of cannula 201. Following, the insertion device 200 is withdrawn from the patient by moving the device 200 in a direction that is roughly parallel to the nasal cavity.

After insertion, the dilator 120 remains in the sinus ostium and expands over a period of time, e.g., 0.5 to 2 hours. Once fully expanded, or at a later time if more convenient for the physician or the patient, the dilator is removed from the enlarged ostium using either forceps or by pulling on tether 129.

As discussed above, the insertion device 200 can be used to insert either the maxillary sinus ostium dilator 100, the sphenoid sinus ostium dilator 120, or both. This is because mounting member or arm 112 has the same size and shape as mounting member or arm 132. Because fin 116 (of dilator 100) has the same size and shape as fin 134 (of dilator 120), and because fin 117 (of dilator 100) has the same size and shape as fin 133 (of dilator 120), the respective dilators can each be mounted on the distal end of cannula 201, although at a 180° rotation one from the other due to the two differently shaped fins in each of the dilators. However, because of the tapered cylindrical shape of handle 202 and the circumferential design of trigger 203, the device is easily rotated into whichever position the user desires while performing the insertion procedure, and so the 180° rotational difference between the mountings of dilators 100 and 120 on insertion device 200 is inconsequential.

In certain embodiments, device 200 includes a light source (not shown in the figures), which in some instances is a directional light source, such as a fiber optic light source, a laser (e.g., a low energy laser), and the like. The light source emits light into the lumen of cannula 201 using known light directing means and a light-reflecting interior surface of cannula 201. In some embodiments, rod 205 and dilator 100 (or 120) are also constructed of light transmitting and/or translucent materials so that the light from the light source causes at least portions of the dilator 100 (or 120) to become illuminated. The illumination may have sufficient intensity so that the emitted light can be seen through the patient's facial tissue. Observing the position of the illuminated dilator 100 (or 120) may help the physician to correctly position the dilator in the ostium of a paranasal sinus.

As can be appreciated from the disclosure provided above, the present disclosure has a wide variety of applications. Accordingly, the following examples are offered for illustration purposes and are not intended to be construed as a limitation on the invention in any way. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### EXAMPLES

### Example 1

An osmotic driver for a paranasal sinus dilator was made as follows. A tube of 304 stainless steel having an outside diameter of 0.64 mm (25 mils) and an inside diameter of 0.51 mm (20 mils) was cut to a length of 1.3 cm and de-burred. An extruded polymer tube (inner membrane) composed of Tecophilic HP-93A-100 polyurethane with an inside diameter slightly larger than that of the steel tube and having a wall thickness of 0.13 mm (5 mils) was slid over the steel tube and cut flush with the length of the steel tube. Next a distal osmotic tablet was fabricated according to the procedures described in Example 1 of U.S. Application Publication No. 2012/0053567, published Mar. 1, 2012, except as follows. First, Polyox 303 was sifted through a 100-mesh sieve. 8.5 g of minus 100 mesh material was transferred to a beaker. Sodium chloride powder was ground with a pestle in a mortar and sifted through a 100-mesh sieve. 15.0 g of sized sodium chloride was added to the Polyox. Next, Methocel E5 was sifted through a 100-mesh sieve. 1.25 g of the sized Methocel was added to the Polyox and sodium chloride. The resulting composition was stirred with a spatula to form a homogenous blend. 7 ml of denatured anhydrous ethanol was slowly stirred into the blend to form a homogenous damp mass. The mass was passed through a 40 mesh sieve with a spatula to form granules. The resulting granules were transferred to a beaker and dried overnight in forced air at 40° C. The dried granules were then sized again through the 40 mesh sieve and transferred to a screw-capped jar. An amount of magnesium stearate equal to 1 percent of the mass of the dried composition was weighed, sized through an 80-mesh sieve, and tumble mixed into the blend for two minutes. Portions of the resulting granulation having a nominal weight of 19.5 mg were compacted into annular shaped tablets using 60 pounds force with a tablet press having flat-faced beveled round tooling with an outside diameter of 2.7 mm and an inside diameter of 0.97 mm. This produced osmotic distal tablets having a nominal length of 2.5 mm. Next, a single distal tablet was slid over the inner member and centered. Next a proximal osmotic tablet composition was prepared using the same procedures and compositions except that the mass of Polyox was 10.98 g and the mass of sodium chloride was 12.5 g. Additionally, 30 mg of yellow ferric oxide pigment, previously sized to minus 100 mesh, was included in the blend during the wet granulation step. The resulting granulation was compacted with the same tablet press and tooling to produce proximal osmotic tablets having a nominal length of 2.5 mm. A single proximal tablet was then slid over the extruded polymeric tube on the steel tube and abutted flush with the distal osmotic tablet. Next, two polymeric extruded pieces (end caps) made of 100% Tecophilic HP-93A-100 with an inside diameter of 0.97 mm (38 mils), a wall thickness of 0.86 mm (34 mils), and a length of 1.5 mm were slid over the distal and proximal ends of the extruded polymeric tube that covers the steel tube and abutted against the osmotic tablets. Next, an extruded polymeric tube (elastic semipermeable outer membrane) composed of 100% Tecophilic HP-93A-100 with an inside diameter of 2.72 mm (107 mils) and a wall thickness of 0.30 mm (12 mils) was slid over the end caps and the osmotic tablets and was cut flush with the outside edges of the end caps so that the outer membrane covered all of the osmotic tablets and end caps. The assembly was then slid distally so that the distal end cap was flush to the distal end of the steel tube. The excess inner member was roll cut off the steel tube. Next, the assembly was covered with heat shrink and the heat shrink was shrunk down over the assembly using heat. Then the outer membrane, end caps, and inner membrane on both the distal and proximal sides of the assembly were bonded together by melting the components together using compression and heat.

### Example 2

A maxillary sinus dilator was made as follows. Taking first an osmotic driver as described in Example 1, a proximal end piece having the same design and shape as end piece 107 shown in FIGS. 3, 4 and 5 was mounted on the proximal end of the driver. The end piece was comprised of injection molded acrylonitrile butadiene styrene having a pocket for the proximal end of the osmotic driver assembly to nest in, a through hole in the center of the pocket for the steel tube to go through, and an angled mounting arm having an angle θ of 110°, was slid over the proximal end of the thermally bonded assembly steel tube until it protruded through the end piece and the proximal end of the osmotic driver was nested inside the end piece pocket. The end piece was affixed to the osmotic driver using adhesive. The protruding steel tube was then mechanically flared against the end piece to create a mechanical stop. Next a stainless steel wire having a length of 1.2 cm (475 mils), a diameter of 0.38 mm (15 mils) and a spherically-shaped distal end having a diameter of 0.89 mm (35 mils) was slid through the axial passageway of an injection molded tapered tip (the tip had the same size and shape as tip 106 shown in FIGS. 3, 4 and 5) made of Pebax until the “ball” end of the wire abutted against the tip. The tip and wire assembly was then slid, wire first, into the distal end of the steel tube of the osmotic driver assembly until the tip abutted against the distal end of the osmotic driver. The tip assembly was then affixed to the osmotic driver using adhesive.

### Example 3

A sphenoid sinus dilator was made as follows. Taking first an osmotic driver as described in Example 1, a proximal end piece having the same design and shape as end piece 127 shown in FIGS. 6, 7 and 8 was mounted on the proximal end of the driver. The end piece was comprised of injection molded acrylonitrile butadiene styrene having a pocket for the proximal end of the osmotic driver assembly to nest in, a through hole in the center of the pocket for the steel tube to go through, and an angled mounting arm having an angle α of 15°, was slid over the proximal end of the thermally bonded assembly steel tube until it protruded through the end piece and the proximal end of the osmotic driver was nested inside the end piece pocket. The end piece was affixed to the osmotic driver using adhesive. The protruding steel tube was then mechanically flared against end piece to create a mechanical stop. Next a stainless steel wire having a length of 1.2 cm (475 mils), a diameter of 0.38 mm (15 mils) and a spherically-shaped distal end having a diameter of 0.89 mm (35 mils) was slid through the axial passageway of an injection molded tapered tip (the tip had the same size and shape as tip 106 shown in FIGS. 3, 4 and 5) made of Pebax until the “ball” end of the wire abutted against the tip. The tip and wire assembly was then slid, wire first, into the distal end of the steel tube of the osmotic driver assembly until the tip abutted against the distal end of the osmotic driver. The tip assembly was then affixed to the osmotic driver using adhesive.

The preceding merely illustrates the principles of the disclosure. All statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, e.g., any elements developed that perform the same function, regardless of structure. The scope of the present disclosure, therefore, is not intended to be limited to the exemplary embodiments shown and described herein. Rather, the scope and spirit of present disclosure is embodied by the appended claims.

1. A device for dilating a stenotic opening of a paranasal sinus in a subject, the device comprising:
(a) a self-expanding driver having an axis and configured to expand from a non-expanded configuration to an expanded configuration, wherein the non-expanded configuration is sized to be positioned within the stenotic sinus opening and wherein the device is sized to be introduced through a nostril opening and a nasal cavity of the subject in order to be positioned in the stenotic sinus opening; and
(b) an arm, extending from a proximal end of the device, for removably mounting the device on an insertion tool, the arm having an axis that approximately aligns with the nasal cavity and the nostril opening when the device is positioned in the stenotic sinus opening.

2. The device of claim 1, wherein the stenotic paranasal sinus opening is a maxillary sinus opening, and the arm axis and the driver axis form an angle in the range of 90° to 130°.

3. The device of claim 2, wherein the angle is in the range of 100° to 120°.

4. The device of claim 2, wherein the angle is 110°.

5. The device of claim 2, wherein the arm has a sufficient length to prevent the device from passing completely through the stenotic opening and into the maxillary sinus.

6. The device of claim 1, wherein the stenotic paranasal sinus opening is a sphenoid sinus opening, and the arm axis and the driver axis form an angle in the range of 0° to 30°.

7. The device of claim 6, wherein the angle is 15°.

8. The device of claim 1, wherein the device has a distal-most to proximal-most length of 10 to 17 mm.

9. The device of claim 1, wherein the arm has a length of 5 mm or more.

10. The device of claim 1, wherein the arm has a circular cross-section for slidably mounting on a cannula of the insertion tool.

11. The device of claim 10, wherein the arm and the cannula have concentric circular cross-sections.

12. The device of claim 10, wherein the arm comprises at least one retention element configured to prevent the arm from rotating within the cannula.

13. The device of claim 12, wherein the retention element is a ridge, a fin or a bump.

14. The device of claim 1, comprising a tether attached to the device for removing the device from the stenotic opening.

15. The device of claim 1, wherein the driver is configured to expand to a diameter of 7 mm or less.

16. The device of claim 1, wherein the driver is configured to expand from the non-expanded configuration to the expanded configuration over a period of 0.5 hours or more.

17. The device of claim 16, wherein the driver is configured to expand from the non-expanded configuration to the expanded configuration over a period of 0.5 to 2 hours.

18. The device of claim 1, wherein the driver is configured to expand by osmosis.

19. The device of claim 1, wherein the driver comprises an osmotically active agent.

20. The device of claim 19, comprising an expandable portion disposed peripherally around the driver.

21. The device of claim 20, wherein the expandable portion comprises an elastic semipermeable membrane.

22. The device of claim 1, wherein the driver exerts an expansion pressure on the stenotic opening of 20 psi or more.

23. A kit comprising the device of claim 1 and a tool for inserting the device into a stenotic sinus opening of a patient.

24. The kit of claim 23, wherein the tool comprises a cannula sized and configured to be removably coupled to the device by inserting at least a portion of the arm into the cannula.

25. The kit of claim 24, wherein the tool comprises:
a handheld member comprising a handle and a trigger;
the cannula having a proximal end coupled to the handheld member and a distal end; and
an interior elongated member coupled to the trigger and extending within a central passageway of the cannula;
wherein the distal end of the cannula is sized to accept insertion of the arm thereinto.

26. The kit of claim 25, wherein the interior elongated member is relatively displaceable with respect to the cannula such that upon actuation of the trigger, the interior elongated member is displaced distally within the cannula.

27. The kit of claim 26, wherein the interior elongated member is configured to push the arm out of the cannula and decouple the device from the tool when the interior elongated member is displaced distally within the cannula.

28. The kit of claim 25, wherein the distal end of the cannula is substantially linear.

29. A device for dilating a stenotic opening of a paranasal sinus in a subject, the device comprising:
(a) a self-expanding driver having an axis and configured to expand from a non-expanded configuration to an expanded configuration, wherein the non-expanded configuration is sized to be positioned within the stenotic opening; and
(b) a proximal anchor comprising an arm extending radially from a proximal end of the device, the arm having a linear axis and being sized and configured to (i) be removably coupled to an insertion tool by inserting at least a portion of the arm into a cannula of said insertion tool, and (ii) prevent the device from passing completely through the stenotic opening and into the paranasal sinus, the driver axis and the arm axis forming an angle, wherein
i) the stenotic opening is a maxillary sinus opening and the angle is in the range of 90° to 130°; or
ii) the stenotic opening is a sphenoid sinus opening and the angle is in the range of 0° to 30°.
